(19) United States
(12) Patent Application Publication
(10) Pub. No.: US 2023/0052272 A1
(43) Pub. Date: Feb. 16, 2023

(54) SYSTEMS AND METHODS FOR SUTURING

(71) Applicant: Ergosurgical Group Corp., Santa Maria, PR (US)

(72) Inventors: Luis Jose Almodovar, Santa Maria, PR (US); Mark A. Follman, Glen Rock, NJ (US)

(21) Appl. No.: 17/877,126

(22) Filed: Jul. 29, 2022

Related U.S. Application Data
(60) Provisional application No. 63/226,911, filed on Jul. 29, 2021.

(51) Int. Cl. A61B 17/06; A61L 17/06

(52) U.S. Cl. CPC A61B 17/06066; A61B 17/06166; A61L 17/06; A61B 2017/0608; A61B 2017/06176

(57) ABSTRACT

Disclosed is a suturing needle having a flexible portion extending from a proximal end of the needle to provide flexure of the needle. Various designs for the flexible portion of the suturing needle are provided.

### CROSS REFERENCE TO RELATED APPLICATIONS

This application claims the benefit of U.S. Provisional Pat. Application 63/226,911 filed 29 Jul. 2021; which is herein fully incorporated by reference for all purposes.

### TECHNICAL FIELD

The present disclosure generally relates to suture needles having a flexible portion.

### BACKGROUND

Suturing procedures used to close wounds or incisions consume a considerable amount of time during a surgical procedure. Generally, the instruments used in suturing procedures are the suturing material, the suturing needle, and the suturing driver. Efforts made to reduce the time and enhance the safety of the procedure have mainly focused on modifications to the needle driver.

The traditional way of driving a needle has been to clamp it down between two flat tips of a needle driver. This forms a static relationship between the needle and needle driver because the needle, once clamped down between the flat tips, cannot move relative to the driver. Moreover, needle drivers are generally designed to fit right-handed users. Thus, left-handed users have difficulties performing the suturing procedure. The right handedness of those devices further affects the capacity of left-handed surgeons to lock and unlock the drivers' locking mechanism. This increases the risks of negative outcomes for patients from undesirable needle driver maneuverings.

### SUMMARY

Recent advances in needle drivers address these deficiencies, including at least this static relationship of the needle and the handedness of the prior art drivers.

Current suture needles have not been designed for, and therefore cannot optimally perform, this task because they were not designed to work with new roller needle drivers. When trying to drive standard swaged needles with these new ergonomic rotational action needle drivers, the rollers generally need to be opened to move over the swaged end of the needle. That is, the swage represents a step or lip on the needle that may be damaged by the rollers, or conversely, that may damage the rollers.

Accordingly, new needle designs that may enable smooth loading of a swaged needle by these new needle drivers are desirable and an object of the present disclosure.

All references, including any patents or patent applications cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents form part of the common general knowledge in the art.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning - i.e. that it will be taken to mean an inclusion of not only the listed components it directly references, but also other non-specified components or elements. This rationale will also be used when the term ‘comprised’ or ‘comprising’ is used in relation to one or more steps in a method or process.

The present disclosure provides a suturing needle comprising a needle tip portion having a needle tip at a distal end for piercing tissue, a needle body having a distal end connected to a proximal end of the needle tip portion, and a flexible portion having a distal end connected to a proximal end of the needle body, wherein the suturing needle comprises a radius, and wherein the suturing needle comprises a single wire having an axis. In certain embodiments the flexible portion comprises a plurality of wedge cuts. In some embodiments the wedge cuts face toward the radius of the suturing needle. In other embodiments the wedge cuts face toward the radius of the suturing needle and away from the radius of the suturing needle. In particular embodiments the flexible portion comprises a plurality of scalloped cuts. In further embodiments the scalloped cuts face toward the radius of the suturing needle. In yet further embodiments the scalloped cuts face toward the radius of the suturing needle and away from the radius of the suturing needle. In additional embodiments the flexible portion comprises a plurality of parallel grooves that are perpendicular to the axis of the wire. In still other embodiments the flexible portion comprises a plurality of helical angular grooves that are at an angle to the axis of the wire.

In some embodiments the suturing needle further comprises a suture thread attached to a proximal end of the flexible portion by a thread attachment component, and a tapered ramp extending from the proximal end of the flexible portion and covering the thread attachment component and at least a portion of the suture thread. In certain embodiments a first end of the tapered ramp proximal the flexible portion has a diameter equal to a needle body diameter, and a second end of the tapered ramp opposite the first end has a diameter equal to a suture thread diameter, and wherein the tapered ramp provides a smooth transition from the first end to the second end thereof. In particular embodiments the tapered ramp is flexible. In further embodiments the tapered ramp is formed of a biocompatible polymer or material. In yet further embodiments the tapered ramp is formed of a cyanoacrylate.

In certain embodiments the needle tip portion is formed to become gradually thicker from the needle tip to the proximal end thereof. In some embodiments the suture thread is mono-filamentary or multi-filamentary. In other embodiments the suture thread is bioabsorbable. In additional embodiments the suture thread comprises at least one region of tissue retaining structures. In further embodiments the tissue retaining structures are barbs. In yet other embodiments the at least one region of tissue retaining structures are distal from the tapered ramp.

The present disclosure also provides a method of suturing tissues using a roller needle driver, the method comprising: (a) providing a suturing needle comprising: a needle tip portion having a needle tip at a distal end for piercing tissue; a needle body having a distal end connected to a proximal end of the needle tip portion; a flexible portion having a distal end connected to a proximal end of the needle body; a suture thread attached to a proximal end of the flexible portion by a thread attachment component; and a tapered ramp extending from the proximal end of the flexible portion and covering the thread attachment component and at least a portion of the suture thread; (b) grasping, between rollers of the roller needle driver, the suture thread or the tapered ramp distal from the flexible portion; (c) loading the needle so that the rollers are positioned on the needle body without opening the rollers; (d) driving the needle through a tissue being subjected to suture; (e) moving the needle through the tissue without releasing the needle from the tissue; (f) grasping the suture needle at an end exiting from the tissue through which the needle was moved; and repeating steps (d)-(f) until completion of the suturing process.

According to certain embodiments, a first end of the needle extension with a tapered transition portion proximal to the needle body has a diameter approximately equal to or slightly larger than or slightly smaller than a needle body diameter, and a second end of the needle extension with a tapered transition portion opposite the first end has a diameter equal to or slightly larger than a suture thread diameter, and wherein the tapered transition portion provides a smooth transition from the first end to the second end thereof. The length of the tapered transition portion may vary to comprise the entirety of the needle extension or just the proximal terminus (trailing end) of the needle extension. The needle extension with a tapered transition portion may be flexible. The needle extension with a tapered transition portion may be formed of a metal or metallic alloy, any suitable polymer or co-polymer, a plastic, a cyanoacrylate, or any other suitable biocompatible material. The needle extension with a tapered transition portion may comprise a tubular portion of any cross-sectional geometry (circular, oval, square, rectangular, triangular, trapezoidal, D-shaped, pentagonal, hexagonal, octagonal, polygonal, star-shaped, gear-shaped, or any other irregular or complex shape). Said tubular section may be a complete tube or may be open in one or more sides to yield a U-shaped cross-section, parentheses cross-section or other cross-sectional shapes. The tapered transition portion at the proximal end of the needle extension may gradually decrease in diameter to form a conical structure where it transitions to the suture. The tapered transition portion at the proximal end of the needle extension may decrease in diameter in a stepwise fashion rather than in the form of a smooth continuous tapered slope. In addition, the tapered transition portion at the proximal end of the needle extension may be straight and taper more abruptly towards its proximal end. Alternatively, the tapered transition portion at the proximal end of the needle extension may taper to form a flattened planar structure (akin to the back part of a toothpaste tube or similar) or it may taper to form a bullet shape rather than a cone.

According to certain embodiments, the needle body may be curved or straight. Moreover, the needle tip portion is formed to become gradually thicker from the needle tip to the proximal end thereof. The needle tip may be sharp or blunt.

According to certain embodiments, the needle tip portion and curved trunk portion are formed of a steel wire, a martensitic stainless steel, or an austenitic stainless steel, or any other suitable biocompatible material. According to certain embodiments, one or more of the foregoing materials form the entire suture needle. In certain embodiments, different portions of the suture needle are formed from different ones or combinations of the foregoing materials. In certain embodiments, one or more of the foregoing materials form a portion of the suture needle. In certain embodiments, the entire needle, including the tip, curved portion, shank, flexible extension, needle extension portion, suture attachment area, and suture attachment portion, is made from any suitable biocompatible material, including nitinol, stainless steel, any other metal or metal alloy, any polymer or any other material suitable for biomedical applications.

According to certain embodiments, the needle tip portion, the curved trunk portion, and/or the entire suture needle may be non-hollow. According to certain embodiments, the needle tip portion, the curved trunk portion, and/or the entire suture needle may be hollow.

According to certain embodiments, the needle body may have a substantially circular cross-section having a non-smooth profile. In one or more embodiment(s), the suturing needle comprises at least one of an oval, a triangular, a square, a rectangular, a pentagonal, a hexagonal, a heptagonal, an octagonal, a nonagonal, a decagonal, or a polygonal having more than ten sided, cross-section. In one or more embodiment(s), the suturing needle comprises multiple varied cross sections, including any combinations of the foregoing cross-sections. The non-smooth surface may comprise 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, about 150, about 200, about 250, about 300, about 350, about 400, about 450, or about 500 equally spaced circumferential indentations forming ridges. In one or more embodiment(s) the non-smooth surface may comprise 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, about 150, about 200, about 250, about 300, about 350, about 400, about 450, or about 500 non-equally spaced indentations forming ridges, or any number in between. In one or more embodiments, the non-smooth surface may comprise milli-indentations, micro-indentations, or nano-indentations. In one or more embodiments, the ridges have a convex shape. In one or more embodiments, the ridges have a flat peak, a pointed peak, a rounded peak, or a bumpy peak. In one or more embodiments, the peaks are smooth, rough, textured, ribbed, tacky, or polished.

According to certain embodiments, the circumferential indentations may be angled on the needle body to form a spiral thereon. The spiral may be right-handed or left-handed. Moreover, the needle body may comprise at least two regions of circumferential indentations having different angles on the curved needle body.

According to certain embodiments, the suture thread may be mono-filamentary or multi-filamentary. The suture thread may be non-bioabsorbable or bioabsorbable. The suture thread may comprise at least one region of tissue retaining structures, such as barbs. According to certain embodiments, the at least one region of tissue retaining structures may be distal from the tapered ramp.

The present disclosure further provides a method of suturing tissues using a rotational needle driver, the method comprising: (a) providing a suture needle comprising: a needle tip portion having a needle tip at a distal end for piercing tissue; a needle body having a distal end connected to a proximal end of the needle tip portion; a suture thread attached to either a proximal end of the needle body by a thread attachment component or attached to any portion of the needle extension with a tapered transition from the distal end of the needle extension to its proximal end where the tapered transition of the needle extension is located; and a needle extension with a tapered transition portion extending from the proximal end of the needle body and covering the thread attachment component and at least a portion of the suture thread (when and if the suture thread is attached to the proximal end of the needle) or not covering the suture thread if said suture thread is attached to the tapered transition portion of the needle extension; (b) grasping, between rollers of the roller needle driver, the suture thread or the needle extension with a tapered transition portion distal from the needle body; (c) loading the needle so that the rollers are positioned on the needle body without opening the rollers; (d) driving the needle through the tissues being subjected to suture; (e) moving the needle through the tissues without releasing the needle from the tissue; (f) grasping the suture needle at an end opposite from an end grasped in step (b); and repeating steps (d) - (f) until completion of the suturing process.

The system as described herein, both as to its configuration and its mode of operation will be best understood, and additional objects and advantages thereof will become apparent, by the following detailed description of a preferred embodiment taken in conjunction with the accompanying drawing.

When the word “invention” is used in this specification, the word “invention” includes “inventions”, that is, the plural of “invention”. By stating “invention”, the Applicant does not in any way admit that the present application does not include more the one patentable and non-obviously distinct invention and Applicant maintains that the present application may include more than one patentably and non-obviously distinct invention. The Applicant hereby asserts, that the disclosure of the present application may include more than one invention, and, in the event that there is more than one invention, that these inventions may be patentable and non-obvious one with respect to the other.

Further, the purpose of the accompanying abstract is to enable the U.S. Patent and Trademark Office and the public generally, and especially the scientists, engineers, and practitioners in the art who are not familiar with patent or legal terms or phraseology, to determine quickly from a cursory inspection the nature and essence of the technical disclosure of the application. The abstract is neither intended to define the full scope of the application, which is measured by the claims, nor is it intended to be limiting as to the scope of the disclosure in any way.

### BRIEF DESCRIPTION OF THE DRAWINGS

All of the figures depict preferred embodiments although other embodiments are contemplated, and the present disclosure is not limited to the embodiments shown.

FIG. 1A and FIG. 1B are illustrations of a suture needle having a wedge cut flexible portion according to certain embodiments of the present disclosure. FIG. 1A shows the entire suture needle, and FIG. 1B is an expanded view of the wedge cut flexible portion.

FIG. 2A and FIG. 2B are illustrations of a suture needle having a scalloped cut flexible portion according to certain embodiments of the present disclosure. FIG. 2A shows the entire suture needle, and FIG. 2B is an expanded view of the scalloped cut flexible portion.

FIG. 3A and FIG. 3B are illustrations of a suture needle having a dual-sided wedge cut flexible portion according to certain embodiments of the present disclosure. FIG. 3A shows the entire suture needle, and FIG. 3B is an expanded view of the dual-sided wedge cut flexible portion.

FIG. 4A and FIG. 4B are illustrations of a suture needle having a dual-sided scalloped cut flexible portion according to certain embodiments of the present disclosure. FIG. 4A shows the entire suture needle, and FIG. 4B is an expanded view of the dual-sided scalloped cut flexible portion.

FIG. 5A and FIG. 5B are illustrations of a suture needle having a flexible portion with parallel grooves according to certain embodiments of the present disclosure. FIG. 5A shows the entire suture needle, and FIG. 5B is an expanded view of the flexible portion with parallel grooves.

FIG. 6A and FIG. 6B are illustrations of a suture needle having a flexible portion with helical angular grooves according to certain embodiments of the present disclosure. FIG. 6A shows the entire suture needle, and FIG. 6B is an expanded view of the flexible portion with helical angular grooves.

FIG. 7 is an illustration of a suture needle having a flexible portion and a needle extension with a tapered transition portion according to certain embodiments of the present disclosure.

### DETAILED DESCRIPTION

To provide an overall understanding of the disclosure, certain illustrative embodiments and examples will now be described. However, it will be understood by one of ordinary skill in the art that the same or equivalent functions and sequences may be accomplished by different embodiments that are also intended to be encompassed within the spirit and scope of the disclosure. The compositions, apparatuses, systems and/or methods described herein may be adapted and modified as is appropriate for the application being addressed and that those described herein may be employed in other suitable applications, and that such other additions and modifications will not depart from the scope hereof.

As used in the specification and claims, the singular forms “a”, “an” and “the” include plural references unless the context clearly dictates otherwise. For example, the term “material” may include a plurality of materials unless the context clearly dictates otherwise. As used in the specification and claims, singular names or types referenced include variations within the family of said name unless the context clearly dictates otherwise.

Certain terminology is used in the following description for convenience only and is not limiting. The words "lower," "upper," "bottom," "top," "front," "back," "left," "right" and "sides" designate directions in the drawings to which reference is made, but are not limiting with respect to the orientation in which the various parts of the suture needle or any assembly of them may be used.

Swaged suturing needles typically comprise attachment of a suture thread to an end of a needle body. These needles have been designed for use with standard needle drivers, which clamp the needle between two flat tips and hold the needle in a static position.

A new generation of devices that move the needle through the tissues by the coordinated rotation of two rollers provide a dynamic relationship between the needle and the needle driver, even when the needle is clamped between the rollers. One of the great advantages of this dynamic relationship is that the needle can be driven in multiple planes of rotation, such as perpendicular to the long axis of the driver with the tip pointing up or down (standard), parallel with the long axis of the driver with the tip pointing straight back towards the back of the device or straight forward away from the device (non-standard), and all angles in between. This dynamic relationship is desirable for the surgeon as it enables placement of the needle at non-traditional angles, permitting suturing of difficult anatomical regions or situations, such as restricted, deep and less accessible locations.

According to certain embodiments, a needle comprising a needle tip portion having a needle tip at a distal end for piercing tissue, a straight or curved trunk portion having a distal end connected to a proximal end of the needle tip portion, a needle extension with or without a tapered transition; and a thread attachment portion connected to a proximal end of the trunk portion or to the proximal end of a needle extension with a tapered transition for connecting a suturing thread, wherein the needle tip portion is formed to become gradually thicker from the needle tip to the proximal end thereof, and wherein the trunk portion is formed to have a substantially circular cross-section smooth profile. In certain embodiments, the needle may have a non-smooth profile. In one or more embodiment(s), the needle comprises at least one of a circular, an oval, an ellipsoidal, an oblong, a semi-circular, a triangular, a square, a rectangular, a pentagonal, a hexagonal, a heptagonal, an octagonal, a nonagonal, a decagonal, or a polygonal having more than ten sided, U-shaped, S-shaped, star-shaped, gear-shaped or any complex or irregular geometrical shape cross-section. In one or more embodiment(s), the suturing needle comprises multiple varied cross sections, including any combinations of the foregoing cross-sections.

Some of these suture needles can be used for medical purposes (e.g., suturing). For example, some of these suture needles can be used in transcatheter suturing, transcatheter intracardiac (or another body organ) suturing, and other flexible platform applications (e.g., endoscopic suturing, colonoscopic suturing). For example, rigid shaft embodiments, as commonly used for laparoscopic, video-assisted thoracoscopic surgery (VATS) and robotic surgery can be used to fundamentally transform suture-based tissue approximation/anchoring from a laborious task to a swift semi-automated or fully-automated endeavor. For example, some of these suture needles can be used for an open surgery, a minimally invasive surgery, a laparoscopic surgery, or an end effector robotic surgery. As such, the some of these suture needles can be used for manual surgery or automated surgery. Some examples of surgeries where some of these suture needles can be employed include, but are not limited to, laparoscopic surgery, robotic surgery, video-assisted or unassisted thoracoscopic surgery, arthroscopic surgery, natural orifice surgery, endoscopic surgery, gynecologic surgery, cardiac surgery, colorectal surgery, pulmonary surgery, gastric bypass surgery, fetal surgery, hysterectomy surgery, dental surgery, urological surgery, brain surgery, or bariatric surgery, or among many others in human (e.g., between newborn until 120 years old, male, female) or animal (e.g., mammal, birds, fish, land animals) applications.

Note that some of these suture needles can be employed in medical or non-medical settings, whether on an object is animate or inanimate. For example, the object, when animate, can include a tissue, an organ, a body part, whether of human or animal, or others. For example, the tissue can be a muscle tissue, a bone tissue, a nerve tissue, an organ tissue, or others. For example, the object, when inanimate, can include a medical device, a prosthesis, an implantable, a machine, a surgical instrument, or others. For example, some of the non-medical setting can include garment making, fabric stitching, knot applications, sewing, shoe making, or others.

The present figures are shown with a “J” -type one piece suture needle having a flexible portion, although other designs for suture needles having a flexible portion are also within the scope of the present disclosure, as detailed above.

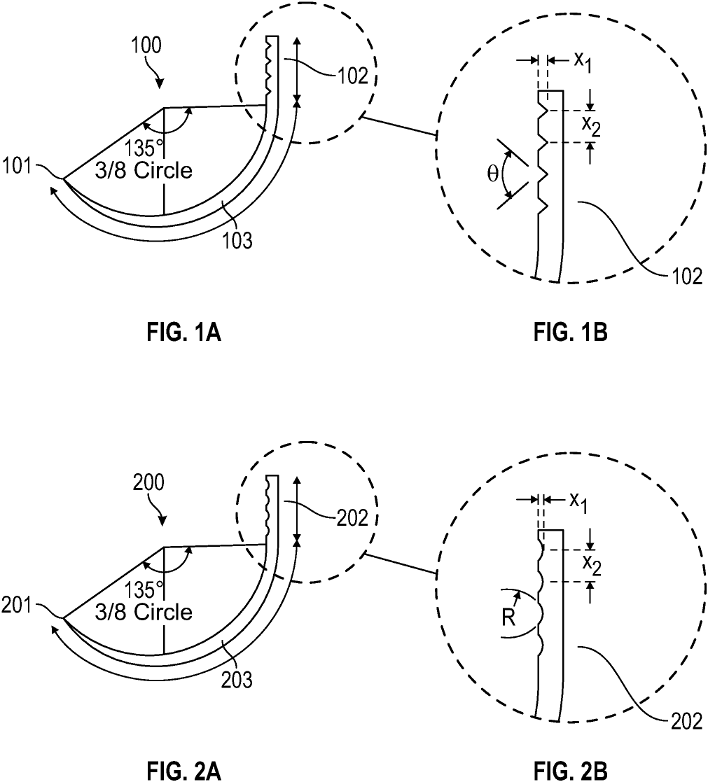

FIG. 1A shows a “J”-type extension needle 100 which is made from a single straight wire. The needle 100 has a needle point 101 needle body 103 and a flexible portion 102. As shown in FIG. 1A, the curved needle is shown as having a round or circular cross-section, in other embodiments the curved needle can also have other cross-sections, including, but not limited to, an oval, an ellipsoidal, an oblong, a semi-circular, a triangular, a square, a rectangular, a pentagonal, a hexagonal, a heptagonal, an octagonal, a nonagonal, a decagonal, or a polygonal having more than ten sided, U-shaped, S-shaped, star-shaped, gear-shaped or any complex or irregular geometrical shape cross-section. As shown in FIG. 1A, the straight flexible portion extension is composed of a plurality of wedge cuts that face the direction of the needle radius. Although the angle (radius) of the needle 100 between the needle tip 101 and the flexible portion 102 is shown in FIG. 1A as about 135°, the angle can be any value from about 0° to about 225° or more, and any value in between, including, but not limited to, about 30°, about 45°, about 60°, about 75°, about 90°, about 105°, about 120°, about 150°, about 165°, about 180°, about 195°, or about 210°. FIG. 1B shows an expanded view of the flexible portion 102 of needle 100. Shown in greater detail are the plurality of wedge cuts having a depth X₁, a distance between the wedge cuts X₂, and an angle of the wedge cuts of theta (θ). Although in FIG. 1A and FIG. 1B angle theta is shown to be about 90°, in various embodiments a number of different angles for theta can be utilized, including, for example, about 45°, about 60°, about 70°, about 105°, about 120° or about 135°. The values for X₁, X₂ and theta can vary depending on the diameter of the wire and the desired flexure or stiffness of the flexible portion. The cuts are processed into the straight wire prior to the application of the needle point and curvature processes. The wedge cuts provide flexure of the needle extension in the direction of the needle curvature. The addition of wedge cuts at 180° from those shown would provide flexure of the needle extension in the opposite direction, but in the same plane, as that of the needle curvature. The continuation of the wedge cuts about the entire circumference of the needle extension would provide flexure in any direction.

FIG. 2A shows a “J”-type extension needle 200 which is made from a single straight wire. The needle 200 has a needle point 201 needle body 203 and a flexible portion 202. As shown in FIG. 2A, the curved needle is shown as having a round or circular cross-section, in other embodiments the curved needle can also have other cross-sections, including, but not limited to, an oval cross-section. As shown in FIG. 2A, the straight flexible portion extension is composed of a plurality of scalloped cuts that face the direction of the needle radius. Although the angle (radius) of the needle 200 between the needle tip 201 and the flexible portion 202 is shown in FIG. 2A as about 135°, the angle can be any value from about 0° to about 225° or more, and any value in between, including, but not limited to, about 30°, about 45°, about 60°, about 75°, about 90°, about 105°, about 120°, about 150°, about 165°, about 180°, about 195°, or about 210°. FIG. 2B shows an expanded view of the flexible portion 202 of needle 200. Shown in greater detail are the plurality of scalloped cuts having a depth X₁, a distance between the scalloped cuts X₂, and a radius of the scalloped cuts of R. Although in FIG. 2A and FIG. 2B the scalloped cuts are shown to have a round or circular cross-section, in various embodiments a number of different profiles for the scalloped cuts can be utilized, including, but not limited to, an oval, an ellipsoidal, an oblong, a semi-circular, a triangular, a square, a rectangular, a pentagonal, a hexagonal, a heptagonal, an octagonal, a nonagonal, a decagonal, or a polygonal having more than ten sided, U-shaped, S-shaped, star-shaped, gear-shaped or any complex or irregular geometrical shape cross-section. The values for X₁, X₂ and R can vary depending on the diameter of the wire and the desired flexure or stiffness of the flexible portion. The cuts are processed into the straight wire prior to the application of the needle point and curvature processes. The scalloped cuts provide flexure of the needle extension in the direction of the needle curvature. The addition of scalloped cuts at 180° from those shown would provide flexure of the needle extension in the opposite direction, but in the same plane, as that of the needle curvature. The continuation of the scalloped cuts about the entire circumference of the needle extension would provide flexure in any direction.

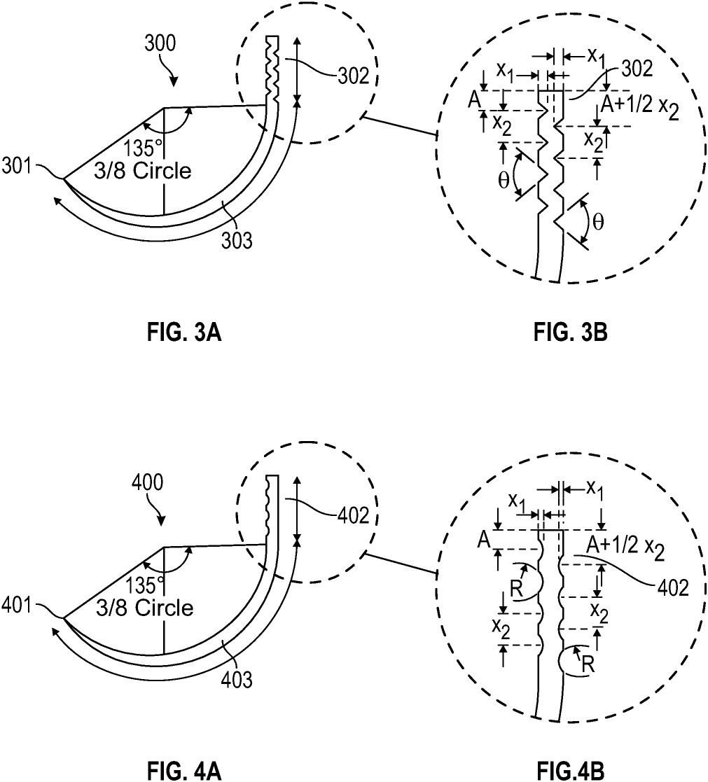

FIG. 3A shows a “J”-type extension needle 300 which is made from a single straight wire. The needle 300 has a needle point 301 needle body 303 and a flexible portion 302. As shown in FIG. 3A, the curved needle is shown as having a round or circular cross-section, in other embodiments the curved needle can also have other cross-sections, including, but not limited to, an oval, an ellipsoidal, an oblong, a semi-circular, a triangular, a square, a rectangular, a pentagonal, a hexagonal, a heptagonal, an octagonal, a nonagonal, a decagonal, or a polygonal having more than ten sided, U-shaped, S-shaped, star-shaped, gear-shaped or any complex or irregular geometrical shape cross-section. As shown in FIG. 3A, the straight flexible portion extension is composed of a plurality of wedge cuts on opposite sides of the flexible portion that are cut on the same plane as the needle radius. Although the angle (radius) of the needle 300 between the needle tip 301 and the flexible portion 302 is shown in FIG. 3A as about 135°, the angle can be any value from about 0° to about 225° or more, and any value in between, including, but not limited to, about 30°, about 45°, about 60°, about 75°, about 90°, about 105°, about 120°, about 150°, about 165°, about 180°, about 195°, or about 210°. FIG. 3B shows an expanded view of the flexible portion 302 of needle 300. Shown in greater detail are the plurality of wedge cuts on opposite sides of the flexible portion having a distance from the end of the needle extension A and A+X₂/2, respectively, a depth X₁, a distance between the wedge cuts X₂, and an angle of the wedge cuts of theta (θ). Although in FIG. 3A and FIG. 3B angle theta is shown to be about 90°, in various embodiments a number of different angles for theta can be utilized, including, for example, about 45°, about 60°, about 70°, about 105°, about 120° or about 135°. The values for A, X₁, X₂, X₂/2 and theta can vary depending on the diameter of the wire and the desired flexure or stiffness of the flexible portion. The cuts are processed into the straight wire prior to the application of the needle point and curvature processes. The wedge cuts provide flexure of the needle extension in the plane of the needle radius.

FIG. 4A shows a “J”-type extension needle 400 which is made from a single straight wire. The needle 400 has a needle point 401 needle body 403 and a flexible portion 402. As shown in FIG. 4A, the curved needle is shown as having a round or circular cross-section, in other embodiments the curved needle can also have other cross-sections, including, but not limited to, an oval, an ellipsoidal, an oblong, a semi-circular, a triangular, a square, a rectangular, a pentagonal, a hexagonal, a heptagonal, an octagonal, a nonagonal, a decagonal, or a polygonal having more than ten sided, U-shaped, S-shaped, star-shaped, gear-shaped or any complex or irregular geometrical shape cross-section. As shown in FIG. 4A, the straight flexible portion extension is composed of a plurality of scalloped cuts on opposite sides of the flexible portion that are cut on the same plane as the needle radius. Although the angle (radius) of the needle 400 between the needle tip 401 and the flexible portion 402 is shown in FIG. 4A as about 135°, the angle can be any value from about 0° to about 225° or more, and any value in between, including, but not limited to, about 30°, about 45°, about 60°, about 75°, about 90°, about 105°, about 120°, about 150°, about 165°, about 180°, about 195°, or about 210°. FIG. 4B shows an expanded view of the flexible portion 402 of needle 400. Shown in greater detail are the plurality of scalloped cuts on opposite sides of the flexible portion having a distance from the end of the needle extension A and A+X₂/2, respectively, a depth X₁, a distance between the scalloped cuts X₂, and an radius of the scalloped cuts of R. Although in FIG. 4A and FIG. 4B the scalloped cuts are shown to have a round or circular cross-section, in various embodiments a number of different profiles for the scalloped cuts can be utilized, including, but not limited to, an oval, an ellipsoidal, an oblong, a semi-circular, a triangular, a square, a rectangular, a pentagonal, a hexagonal, a heptagonal, an octagonal, a nonagonal, a decagonal, or a polygonal having more than ten sided, U-shaped, S-shaped, star-shaped, gear-shaped or any complex or irregular geometrical shape cross-section. The values for A, X₁, X₂, X₂/2 and R can vary depending on the diameter of the wire and the desired flexure or stiffness of the flexible portion. The cuts are processed into the straight wire prior to the application of the needle point and curvature processes. The scalloped cuts provide flexure of the needle extension in the direction of the needle curvature.

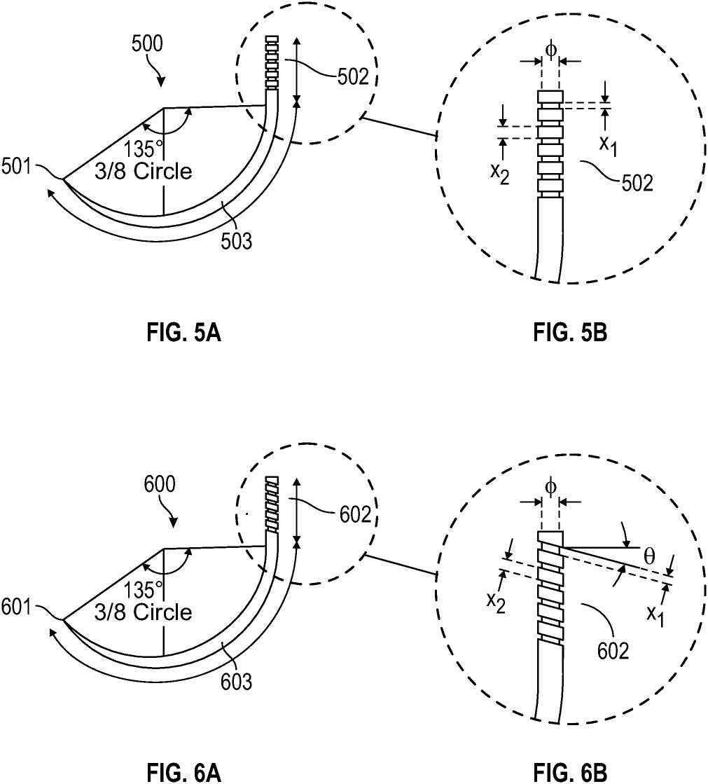

FIG. 5A shows a “J”-type extension needle 500 which is made from a single straight wire. The needle 500 has a needle point 501 needle body 503 and a flexible portion 502. As shown in FIG. 5A, the curved needle is shown as having a round or circular cross-section, in other embodiments the curved needle can also have other cross-sections, including, but not limited to, an oval, an ellipsoidal, an oblong, a semi-circular, a triangular, a square, a rectangular, a pentagonal, a hexagonal, a heptagonal, an octagonal, a nonagonal, a decagonal, or a polygonal having more than ten sided, U-shaped, S-shaped, star-shaped, gear-shaped or any complex or irregular geometrical shape cross-section. As shown in FIG. 5A, the straight flexible portion extension is composed of a plurality of parallel grooves that are cut perpendicular to the axis of the wire. Although the angle (radius) of the needle 500 between the needle tip 501 and the flexible portion 502 is shown in FIG. 5A as about 135°, the angle can be any value from about 0° to about 225° or more, and any value in between, including, but not limited to, about 30°, about 45°, about 60°, about 75°, about 90°, about 105°, about 120°, about 150°, about 165°, about 180°, about 195°, or about 210°. FIG. 5B shows an expanded view of the flexible portion 502 of needle 500. Shown in greater detail are the plurality of parallel grooves having a width of the grooves X₁, a distance between the grooves X₂, and inner diameter of the grooves of phi (φ). The values for X₁, X₂ and phi (φ) can vary depending on the outer diameter of the wire and the desired flexure or stiffness of the flexible portion. The parallel groove cuts are processed into the straight wire prior to the application of the needle point and curvature processes. The parallel groove cuts provide flexure of the needle extension in all planes, as needed during some suturing applications.

FIG. 6A shows a “J”-type extension needle 600 which is made from a single straight wire. The needle 600 has a needle point 601 needle body 603 and a flexible portion 602. As shown in FIG. 6A, the curved needle is shown as having a round or circular cross-section, in other embodiments the curved needle can also have other cross-sections, including, but not limited to, an oval, an ellipsoidal, an oblong, a semi-circular, a triangular, a square, a rectangular, a pentagonal, a hexagonal, a heptagonal, an octagonal, a nonagonal, a decagonal, or a polygonal having more than ten sided, U-shaped, S-shaped, star-shaped, gear-shaped or any complex or irregular geometrical shape cross-section. As shown in FIG. 6A, the straight flexible portion extension is composed of a plurality of helical angular grooves that are cut at an angle theta (θ) to the axis of the wire. The grooves can be cut in clockwise or counterclockwise directions. Although the angle (radius) of the needle 600 between the needle tip 601 and the flexible portion 602 is shown in FIG. 6A as about 135°, the angle can be any value from about 0° to about 225° or more, and any value in between, including, but not limited to, about 30°, about 45°, about 60°, about 75°, about 90°, about 105°, about 120°, about 150°, about 165°, about 180°, about 195°, or about 210°. FIG. 6B shows an expanded view of the flexible portion 602 of needle 600. Shown in greater detail are the plurality of parallel grooves having a width of the grooves X₁, a distance between the grooves X₂, and inner diameter of the grooves of phi (φ), and an angle of the grooves theta (θ). The values for X₁, X₂, phi (φ) and theta (θ) can vary depending on the outer diameter of the wire and the desired flexure or stiffness of the flexible portion. The helical angular groove cuts are processed into the straight wire prior to the application of the needle point and curvature processes. The helical angular groove cuts provide flexure of the needle extension in all planes, as needed during some suturing applications.

Current swaged suture needles generally include a lip or step-up in the transition from the suture thread to the thread attachment connector, and another lip or step-up in the transition from the thread attachment component to the needle body. Alone or in combination, these transitions can pose a problem for the rollers of a rotational needle driver, as the suture thread may be damaged by the rollers, or conversely, the transitions may damage the rollers of the needle driver. For example, the rollers may roll past the straight flexible portion extension sections described herein and past the thread attachment component of the needle and may clamp down on that region and damage/cut the suture thread.

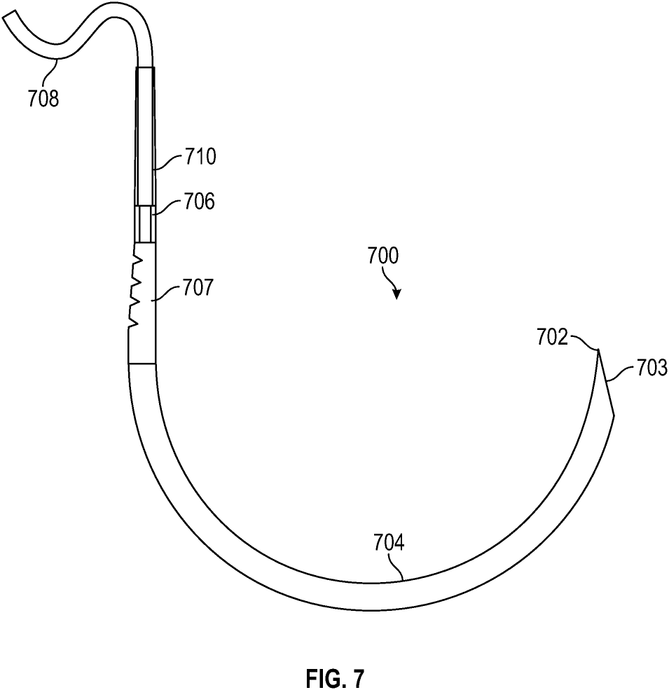

Certain embodiments of the present disclosure overcome these deficiencies by incorporating a tapered transition portion that provides a smooth transition from the suture thread to the needle extension or the needle body. With reference to FIG. 7, a suture needle 700 of the present disclosure may comprise a needle extension with a tapered transition portion 710. This protective leading segment immediately follows the flexible portion 707 and the distal end of the needle body 704, such as covering the thread attachment component 706 of the needle and extends over a proximal portion of the suture thread 708. In certain embodiments, there is no or minimal transition between the flexible portion 707 of the needle and the thread attachment portion 706. In such embodiments, the transition portion 710 provided a taper from the flexible portion 707 and past the thread attachment portion 706. In certain embodiments, the tapered transition portion 710 includes a proximal side that overlaps the flexible portion 707 in a direction of the needle tip and includes a distal side that extends to a point overlapping the suture to form a taper. In certain embodiments, the taper is gradual. In certain embodiments the taper is steep. In certain embodiments, the swage is tapered. In certain embodiments, the swage is non-tapered.

The needle extension with a tapered transition portion 710 may extend over the suture material a distance from the end of the needle body 704 of the needle, such as from about 0.1 cm, about 0.2 cm, about 0.3 cm, about 0.4 cm, about 0.5 cm, about 0.6 cm, about 0.7 cm, about 0.8 cm, about 0.9 cm, about 1 cm, about 2 cm, about 3 cm, about 4 cm, about 5 cm, about 6 cm, about 7 cm, about 8 cm, about 9 cm, about 10 cm, about 15 cm, about 20 cm, or any distance in between. Moreover, the tapered transition portion 710 may be formed of a flexible material that may assist in loading the needle onto the needle driver, but may have more rigidity than the suture material, such as to aid in suture management during knot tying. The length of the needle extension with a tapered transition portion 710 may be determined by the optimum balance between the minimal length needed by the surgeon to grab the needle by the needle extension with a tapered transition when inserting it or withdrawing it to a surgical site, such as through a laparoscopic port, and the maximal length that can feasibly allow an extension of the needle with a tapering transition and enough flexibility to go through tissue following the needle path without causing tissue trauma.

The needle extension with a tapered transition portion 710 may be a cyanoacrylate or another suitable adhesive coating (or filament) material, such as, for example, another biocompatible material, that may be built up from a diameter of the suture material 708 to a diameter of the needle body 704 of the needle 700. The needle extension with a tapered transition portion 710 may be formed by a shrink-wrapped procedure wherein a preform of the needle extension with a tapered region is positioned over the suture thread and swage to abut an end of the trunk portion of the needle, and heat may be applied to form the preform on the needle, thus forming the needle extension with a tapered transition portion 710. As such, this protective segment or needle extension with a tapered transition portion 710 may be shaped as a “conical ramp” that allows the rollers to be rolled from the needle extension with a tapered transition portion 710 “up the ramp” in a continuous rolling motion until reaching the non-tapered portion of the needle extension or the needle body 704 of the needle 700 without hitting the blunt swage end of the needle, and thus avoiding potential damage to the needle, rollers, and/or suture material. In certain embodiments, the needle extension with a tapered transition portion may comprise a magnetic material or a magnetizable material. In certain embodiments, the suture material may comprise a magnetic material or a magnetizable material.

This creates a procedural benefit and efficiency by loading the needle onto the needle driver to a position ready for suturing all in a single step. The rollers may be closed over the suture material 708 and may rotate in opposite directions to advance the needle forward. As the needle advances between the rollers of the needle driver, the rollers smoothly advance the needle from the suture material to the needle extension with a tapered transition portion 710. Without opening the rollers, the rollers may continue to rotate to advance the needle along the needle extension with a tapered transition portion 710 over the thread attachment component (if and when the suture thread is attached to the needle attachment portion of the needle instead of being attached to any point along the length of the needle extension inclusive of the proximal tapered portion of the needle extension) 706.

In the absence of the needle extension with a tapered transition portion 710, this would represent a step or lip on the needle that may be damaged by the rollers, or conversely, that may damage the rollers. Accordingly, in the absence of the needle extension with a tapered transition portion 710, the clinician would have to use a grasper to hold/stabilize the needle, then open the needle driver to release the suture it was grasping during needle insertion and then grasp the needle with the needle driver once positioned over the needle body in preparation for suturing. In a laparoscopic setting each of these steps may require time, effort, coordination and maybe even repetition due to the lack of depth perception (2D visualization systems used mostly) and decreased dexterity, fulcrum effect of port, etc. Finally, the rotation of the rollers advances the needle so that it is positioned between the rollers and properly loaded for suturing without ever opening the rollers. These motions may be reversed to unload the needle, such as to remove the needle from a suture site (e.g., from the abdomen or from a laparoscopic surgical site).

When used with a rotational needle driver, such as discussed above, the rollers may roll past the straight flexible portion extension sections 707 described herein and past the thread attachment portion 706 of the needle 700 and may clamp down on that region and damage/cut the suture thread. Thus, according to certain embodiments, the suture needle of the present disclosure may comprise a needle extension with a tapered transition portion 710. This protective leading segment immediately follows the distal end of the trunk portion 704 of the needle, such as covering the thread attachment portion 706 of the needle(if and when the suture thread is attached to the needle attachment portion of the needle instead of being attached to any point along the length of the needle extension inclusive of the proximal tapered portion of the needle extension), and extends over a proximal portion of the suture material (if and when the suture thread is attached to the needle attachment portion of the needle instead of being attached to any point along the length of the needle extension inclusive of the proximal tapered portion of the needle extension).

With reference to FIG. 7, a suture needle 700 of the present disclosure generally comprises a needle tip 702 for piecing a tissue. The needle tip 702 is positioned at a distal end of a needle tip portion 703, wherein the needle tip portion becomes gradually thicker from the needle tip to a proximal end thereof. Thus, the needle tip potion may be a tapered tip wherein the needle tip portion is round and tapers smoothly to a point. Alternatively, the needle tip portion may be triangular as shown in FIG. 7, and may have a sharpened cutting edge on the inside or on the outside, or may have a “trocar point” or “taper cut” whereby the needle body is round and tapered, but ends in a small triangular cutting point. Alternatively, the needle tip may be a blunt point, such as for suturing friable tissues, or may have a needle tip portion that includes “side cutting” or “spatula points” whereby the needle is flat on top and bottom with a cutting edge along the front to one side (these are typically used for eye surgery).

Continuous with the proximal end of the needle tip portion 703 is the needle body 704 having the thread attachment component 706 positioned at a distal end thereof. The needle body 704 is shown to have a curve in the drawings. Exemplary curves include at least half curved or ski, ¼ circle, ⅜ circle, ½ circle, such as shown in the figures, ⅝ circle, J-shaped, S-shaped, spatula shaped, U-shaped and compound curve. Alternatively, the needle body may be straight, such that the present disclosure may comprise a straight needle.

The material configuring the suture needle is not limited, and may comprise a metal or a metal alloy, such as, for example, a biocompatible metal or metal alloy. In certain embodiments, the suture needle comprises any of steel wire, a martensitic stainless steel, or an austenitic stainless steel. In certain embodiments, the suture needle is comprised of a magnetic or magnetizable material. In certain embodiments, the suture needle is comprised of a nitinol (nickel titanium) material of superelastic or shape memory properties. When formed of steel wire or martensitic stainless steel, the needle may be hardened by thermal treatment. When formed of a nitinol (nickel titanium) material, a martensitic stainless steel, or an austenitic stainless steel, or any other metal or metallic material, the needle may be hardened by thermal treatment or any other known procedure for treating such materials. In certain embodiments, one or more such treatments are applied either to the entirety of the needle inclusive of the flexible extension and suture attachment portion or to any portion of the needle (such as only to the tip, to the body, the shank, the flexible extension or the suture attachment portion) either alone or in any combination or permutation of any of those portions. In certain embodiments, each portion or individual portions of the needle (alone or in any combination or permutation) is treated with any of the foregoing treatments, such as, for example, heat or siliconization. Other processes when forming or finishing the needle may be siliconization, coating with any number or combination of biocompatible coating materials or lubrication with any number or combination of biocompatible lubricating agents, among other processes. In certain embodiments, the entire suture needle is formed from one or more of the foregoing materials. In certain embodiments, one or more of the foregoing materials form a portion of the suture needle. In certain embodiments, different portions of the suture needle are formed from different ones or combinations of the foregoing materials. In certain embodiments, the entire needle, including the needle extension portion and suture attachment portion, is made from any suitable biocompatible material, including nitinol, stainless steel, any other metal or metal alloy, any polymer or any other material suitable for biomedical applications.

The suture thread 708 may be swaged to the needle 700 by the thread attachment component 706. Accordingly, the needle is generally an atraumatic needle, i.e., eyeless needle, having a suture material or thread attached at an end by swaging whereby the suture material is inserted into a channel at the blunt end of the needle, such as into the thread attachment component 706, which is then deformed to a final shape to hold the suture and needle together. Alternatively, the suture thread may be swaged to the needle extension at any point along the length of said needle extension inclusive of the proximal tapered transition portion of said needle extension. The needle, needle extension or tapered transition portion of said needle extension may be permanently swaged to the suture material or may be designed to come off the suture material with a sharp straight tug. These “pop-offs” are commonly used for interrupted sutures, where each suture is only passed once and then tied.

The suture material or thread may be mono-filamentary, i.e., formed of a single filament, or multi-filamentary, i.e., formed from a combination of two or more filaments, e.g., three filaments arranged in a braided fashion. The suture thread has a length, where that length is typically at least 5 inches, or at least 10 inches, or at least 15 inches, or at least 20 inches. The suture thread will typically have two ends, which may be described as a deployment end and/or a trailing end. In such a case, the deployment end of the suture thread is that end that first enters tissue, adjacent to the needle, such as connected via the thread attachment component to the distal end of the needle body of the needle or to any point along the length of a needle extension inclusive of the proximal tapered transition portion of said needle extension. Alternatively, the suture material may be looped, such that each of the two free ends are connected to the needle by the thread attachment component or connected to any point along the length of a needle extension inclusive of the proximal tapered transition portion of said needle extension.

The thread 708 can be a suture, which can be non-absorbable or absorbable of various gauges. The thread 708 can include silk, cotton, fabric, nylon, polyester, silver, copper, Dacron, rubber, silicon, plain or chromic catgut, polyglycolide, polydioxanone, monocryl, polypropylene, triclosan, caprolactone, polymer, glycolide, I-lactide, p-dioxanone, trimethylene carbonate, ε-caprolactone, stainless steel, ceramic, glass, leather, or other natural or artificial materials. The thread 708 is solid, but can be perforated. The thread 708 is internally dense, but can be hollow. The thread 708 can be rigid, semi-rigid, elastic, resilient, or flexible. For example, the thread 708 can bend about 90 degrees or less (e.g., inclusively between or about 90, 80, 70, 60, 50, 40, 30, 20, or 10 degrees) or more (e.g., inclusively between or about 90, 100, 110, 120, 130, 140, 150, 160, 170, or 180 degrees). The thread 708 can have a cross-section that is closed-shaped (e.g., O-shape, D-shape, 0-shape, square, rectangle, triangle, polygon) or open-shaped (e.g., U-shape, C-shape, V-shape), whether symmetrical or asymmetrical. The thread 708 can have barbed protrusions to facilitate tissue fixation without the need for tying knots.

The suture material or thread may be bioabsorbable, such that after introduction into a tissue it is broken down and absorbed by the body. Typically, the degradation process is at least partially mediated by, or performed in, a biological system. Accordingly, bioabsorbable refers to a chain scission process by which a polymer chain is cleaved through various mechanisms, including, for example, by chemical reaction (e.g., hydrolysis, oxidation/reduction, enzymatic mechanisms or a combination of these) or by a thermal or photolytic process. Bioabsorbable suture material may include polymers such as polyglycolic acid, copolymers of glycolide and lactide, copolymers of trimethylene carbonate and glycolide with diethylene glycol (e.g., MAXON™, Tyco Healthcare Group), terpolymer composed of glycolide, trimethylene carbonate, and dioxanone (e.g., BIOSYN™[glycolide (60%), trimethylene carbonate (26%), and dioxanone (14%)], Tyco Healthcare Group), copolymers of glycolide, caprolactone, trimethylene carbonate, and lactide (e.g., CAPROSYN™, Tyco Healthcare Group). In certain embodiments, the bioabsorbable suture material may comprise or include any other polymer useful for suturing applications that currently exists or that may be developed in the future.

Alternatively, the suture material or thread may be non-degradable, such that it is not degraded by chemical, thermal, or photolytic process. Non-degradable suture material includes polyamide (also known as nylon, such as nylon 6 and nylon 6.6), polyester (e.g., polyethylene terephthlate), polytetrafluoroethylene (e.g., expanded polytetrafluoroethylene), polyether-ester such as polybutester (block copolymer of butylene terephthalate and polytetra methylene ether glycol), polyurethane, metal alloys, metal (e.g., stainless steel wire), polypropylene, polyethelene, silk, and cotton. Sutures made of non-degradable suture material are particularly suitable for applications in which the suture is meant to remain permanently or is meant to be physically removed from the body. In certain embodiments, the non-degradable suture material may comprise or include any other polymer useful for suturing applications that currently exists or that may be developed in the future.

The suture material or thread may comprise a coating or agent applied to a surface thereof that may affect would healing, such as a coating material, wound healing agent, antimicrobial agent, antibacterial agent, growth factor, adhesive, sealant, blood product, blood component, preservative, anti-adhesive, protein, polysaccharide, peptide, genetic material, viral vector, nucleic acid, nucleotide, plasmid, lymphokine, radioactive agent, metal, alloy, salt, growth factor, growth factor antagonist, cell, hydrophobic agent, hydrophilic agent, immunological agent, anti-colonization agent, and combinations thereof. The suture material or thread may comprise a coating or agent applied to a surface thereof that may enhance the surgeon’s ability to accurately suture, such as colorants, dyes, ultraviolet absorbers, ultraviolet stabilizers, photochromic agents, diagnostic agent, imaging agent, radiopaque agent, or combinations thereof.

The suture material or thread may have a diameter indicated by a cross-sectional dimension. This diameter may be determined at a location along the suture where there are either no barbs, or the barbs that are present are pushed against the suture body so that they are flush with the surface of the suture body. The suture may have no barbs or may have barbs along all or only a portion of the suture length. According to certain embodiments, the suture may have at least one barbed region positioned distal from the thread attachment component. Such positioning may allow the presently disclosed suture needle to be backed out of a suture site, wherein the suture material adjacent the needle would not have barbs and would easily slide back through the tissue. In some embodiments the needle extension with a tapered transition portion covers the barbs on the suture, thereby allowing the suture needle to be backed out of the suture sight. More distally located barbed suture may remain securely in place once positioned in the tissue.

The suture may have a generally circular cross-sectional shape, or may have a non-circular shape, e.g., polygonal such as 3-sided (triangular), or 4-, 5- or 6-sided (hexagonal) sided. The cross section of the suture body may have an oval, an ellipsoid, an oblong, or a semi-circular appearance.

Suture sizing is based upon diameter. The United States Pharmacopeia (“USP”) designation of suture size runs from 0 to 7 in the larger range and 1-0 to 11-0 in the smaller range; in the smaller range, the higher the value preceding the hyphenated zero, the smaller the suture diameter. Under the USP nomenclature system, the actual diameter of a suture will depend on the suture material, so that, by way of example, a suture of size 5-0 and made of collagen will have a diameter of 0.15 mm, while sutures having the same USP size designation but made of a synthetic absorbable material or a non-absorbable material will each have a diameter of 0.1 mm. The selection of suture size for a particular purpose depends upon factors such as the nature of the tissue to be sutured and the importance of cosmetic concerns; while smaller sutures may be more easily manipulated through tight surgical sites and are associated with less scarring, the tensile strength of a suture manufactured from a given material tends to decrease with decreasing size. It is to be understood that the suture materials for use with the suture needles disclosed herein include without limitation 7, 6, 5, 4, 3, 2, 1, 0, 1-0, 2-0, 3-0, 4-0, 5-0, 6-0, 7-0, 8-0, 9-0, 10-0 and 11-0. It is to be understood that a variety of suture lengths may be used with the suture needles described herein.

The needle body may have any profile known in the art, such as circular, oval, triangular, ellipsoidal, oblong, semi-circular, triangular, square, rectangular, pentagonal, hexagonal, heptagonal, octagonal, nonagonal, decagonal, or polygonal having more than ten sided, U-shaped, S-shaped, star-shaped, gear-shaped or any complex or irregular geometrical shape and the like. The needle body can have a smooth surface. According to certain embodiments, the needle body may be a non-smooth profile. For example, when trying to drive needle through a tissue using a rotational needle drive, a smooth surface of the needle body may allow the needle to deviate out of an initial chosen plane of rotation, especially for needle bodies having an oval shape. Moreover, it is not possible to securely position these needles in a non-standard angle. Accordingly, an object of the present disclosure is to provide suture needles that may perform optimally in roller needle drivers and may incorporate features that enable multi-planar needle driving.

Features or functionality described with respect to certain example embodiments may be combined and sub-combined in and/or with various other example embodiments. Also, different features and/or elements of example embodiments, as disclosed herein, may be combined and sub-combined in a similar manner as well. Further, some example embodiments, whether individually and/or collectively, may be components of a larger system, wherein other procedures may take precedence over and/or otherwise modify their application. Additionally, a number of steps may be required before, after, and/or concurrently with example embodiments, as disclosed herein. Note that any and/or all methods and/or processes, at least as disclosed herein, can be at least partially performed via at least one entity or actor in any manner.

As used herein, a term “about” or “substantially” refers to a +/- 10% variation from a nominal value/term. Such variation is always included in any given value/term provided herein, whether or not such variation is specifically referred thereto.

In particular, the presently described suture needles can be used for an open surgery, a minimally invasive surgery, a laparoscopic surgery, or an end effector robotic surgery. As such, the presently described suture needles can be used for manual surgery or automated surgery. Some examples of surgeries where the presently described suture needles can be employed include laparoscopic surgery, robotic surgery, video-assisted or unassisted thoracoscopic surgery, arthroscopic surgery, natural orifice surgery, endoscopic surgery, gynecologic surgery, cardiac surgery, colorectal surgery, pulmonary surgery, gastric bypass surgery, hysterectomy surgery, fetal surgery, dental surgery, urological surgery, brain surgery, or bariatric surgery, or among many others in human (e.g., between newborn until 120 years old, male, female) or animal (e.g., mammal, birds, fish, land animals) applications. However, note that the suture needle 200 can also be applied to non-medical applications, such as garment making, fabric stitching, knot applications, sowing, shoe making, or others.

Any component described herein can include a material suitable for a medical use. The material can be, flexible, elastic, or resilient. The material can be suitable to be disinfected, sterilized, or sanitized, which can be with a hot steam, an autoclave, or others. For example, the material can include plastic, metal, rubber, shape memory, fabric, foam, or others.

The device and system of the present disclosure has been described with specific reference to certain drawings and various embodiments, but may, however, be embodied in many different forms and should not be construed as necessarily being limited to only embodiments disclosed herein. Rather, these embodiments are provided so that this disclosure is thorough and complete, and fully conveys various concepts of this disclosure to skilled artisans.

Note that various terminology used herein can imply direct or indirect, full or partial, temporary or permanent, action or inaction. For example, when an element is referred to as being “on,” “connected” or “coupled” to another element, then the element can be directly on, connected or coupled to the other element or intervening elements can be present, including indirect or direct variants. In contrast, when an element is referred to as being “directly connected” or “directly coupled” to another element, there are no intervening elements present.

Likewise, as used herein, a term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances.

Similarly, as used herein, various singular forms “a,” “an” and “the” are intended to include various plural forms as well, unless context clearly indicates otherwise. For example, a term “a” or “an” shall mean “one or more,” even though a phrase “one or more” is also used herein.

Moreover, terms “comprises,” “includes” or “comprising,” “including” when used in this specification, specify a presence of stated features, integers, steps, operations, elements, or components, but do not preclude a presence and/or addition of one or more other features, integers, steps, operations, elements, components, or groups thereof. Furthermore, when this disclosure states that something is “based on” something else, then such statement refers to a basis which may be based on one or more other things as well. In other words, unless expressly indicated otherwise, as used herein “based on” inclusively means “based at least in part on” or “based at least partially on.”

Additionally, although terms first, second, and others can be used herein to describe various elements, components, regions, layers, or sections, these elements, components, regions, layers, or sections should not necessarily be limited by such terms. Rather, these terms are used to distinguish one element, component, region, layer, or section from another element, component, region, layer, or section. As such, a first element, component, region, layer, or section discussed below could be termed a second element, component, region, layer, or section without departing from this disclosure.

Also, unless otherwise defined, all terms (including technical and scientific terms) used herein have the same meaning as commonly understood by one of ordinary skill in an art to which this disclosure belongs. As such, terms, such as those defined in commonly used dictionaries, should be interpreted as having a meaning that is consistent with their meaning in a context of a relevant art and should not be interpreted in an idealized or overly formal sense unless expressly so defined herein.

In addition, features described with respect to certain example embodiments may be combined in or with various other example embodiments in any permutational or combinatory manner. Different features or elements of example embodiments, as disclosed herein, may be combined in a similar manner. The term “combination”, “combinatory,” or “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

Although preferred embodiments have been depicted and described in detail herein, skilled artisans know that various modifications, additions, substitutions and the like can be made without departing from spirit of this disclosure. As such, these are considered to be within the scope of the disclosure, as defined in the following claims.

1. A suturing needle comprising:
a needle tip portion having a needle tip at a distal end for piercing tissue;
a needle body having a distal end connected to a proximal end of the needle tip portion; and
a flexible portion having a distal end connected to a proximal end of the needle body;
wherein the suturing needle comprises a radius, and wherein the suturing needle comprises a single wire having an axis.

2. The suturing needle of claim 1, wherein the flexible portion comprises a plurality of wedge cuts.

3. The suturing needle of claim 2, wherein the wedge cuts face toward the radius of the suturing needle.

4. The suturing needle of claim 2, wherein the wedge cuts face toward the radius of the suturing needle and away from the radius of the suturing needle.

5. The suturing needle of claim 1, wherein the flexible portion comprises a plurality of scalloped cuts.

6. The suturing needle of claim 5, wherein the scalloped cuts face toward the radius of the suturing needle.

7. The suturing needle of claim 5, wherein the scalloped cuts face toward the radius of the suturing needle and away from the radius of the suturing needle.

8. The suturing needle of claim 1, wherein the flexible portion comprises a plurality of parallel grooves that are perpendicular to the axis of the wire.

9. The suturing needle of claim 1, wherein the flexible portion comprises a plurality of helical angular grooves that are at an angle to the axis of the wire.

10. The suturing needle of claim 1, further comprising:
a suture thread attached to a proximal end of the flexible portion by a thread attachment component; and
a tapered ramp extending from the proximal end of the flexible portion and covering the thread attachment component and at least a portion of the suture thread.

11. The suturing needle of claim 10, wherein a first end of the tapered ramp proximal the flexible portion has a diameter equal to a needle body diameter, and a second end of the tapered ramp opposite the first end has a diameter equal to a suture thread diameter, and wherein the tapered ramp provides a smooth transition from the first end to the second end thereof.

12. The suturing needle of claim 10, wherein the tapered ramp is flexible.

13. The suturing needle of claim 10, wherein the tapered ramp is formed of a biocompatible polymer or material.

14. The suturing needle of claim 13, wherein the tapered ramp is formed of a cyanoacrylate.

15. The suturing needle of claim 10, wherein the needle tip portion is formed to become gradually thicker from the needle tip to the proximal end thereof.

16. The suturing needle of claim 10, wherein the suture thread is mono-filamentary or multi-filamentary.

17. The suturing needle of claim 10, wherein the suture thread is bioabsorbable.

18. The suturing needle of claim 10, wherein the suture thread comprises at least one region of tissue retaining structures.

19. The suturing needle of claim 18, wherein the tissue retaining structures are barbs.

20. The suturing needle of claim 18, wherein the at least one region of tissue retaining structures are distal from the tapered ramp.

21. The suturing needle of claim 1, wherein at least a portion of the suturing needle comprises at least one of a nitinol (nickel titanium) material, a martensitic stainless steel material, or an austenitic stainless steel material, or any other metal or metallic material.

22. The suturing needle of claim 1, wherein at least a portion of the suturing needle is hardened by at least one of thermal treatment or siliconization.

23. A method of suturing tissues using a roller needle driver, the method comprising:
(a) providing a suturing needle comprising: a needle tip portion having a needle tip at a distal end for piercing tissue; a needle body having a distal end connected to a proximal end of the needle tip portion; a flexible portion having a distal end connected to a proximal end of the needle body; a suture thread attached to a proximal end of the flexible portion by a thread attachment component; and a tapered ramp extending from the proximal end of the flexible portion and covering the thread attachment component and at least a portion of the suture thread;
(b) grasping, between rollers of the roller needle driver, the suture thread or the tapered ramp distal from the flexible portion;
(c) loading the needle so that the rollers are positioned on the needle body without opening the rollers;
(d) driving the needle through a tissue being subjected to suture;
(e) moving the needle through the tissue without releasing the needle from the tissue;
(f) grasping the suture needle at an end exiting from the tissue through which the needle was moved; and
repeating steps (d) - (f) until completion of the suturing process.
